(19) United States
(12) Patent Application Publication
(10) Pub. No.: US 2004/0024461 A1
(43) Pub. Date: Feb. 5, 2004

(54) Spring and spherical joint artificial disc replacements

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/434,931

(22) Filed: May 9, 2003

Related U.S. Application Data
(60) Provisional application No. 60/379,462, filed on May 10, 2002.

(51) Int. Cl. A61F 2/44

(52) U.S. Cl. 623/17.13

(57) ABSTRACT

One or more springs in an artificial disc replacement (ADR) provide natural movement and extend life of the spring or ADR. In the preferred embodiment, the springs articulate with at least one convex or concave surface on an endplate (EP) of the ADR. More particularly, the spring or springs may articulate with, or connect to, concave or convex articulating EP components. In various alternative embodiments, the ADR EPs may include features that impinge or otherwise limit maximum load on a spring or on multiple springs. The springs may be disposed in cylinders, over posts, or otherwise constrained. For example, spring posts with convex or concave surfaces may articulate with corresponding concave or convex components, or concave or convex surfaces, on ADR EPs. Also, the center of rotation (COR) of the preferably spherical joint may vary vertically by compression and expansion of a compressible component. Alternatively, the ADR may include multiple, separate CORs that cooperate simultaneously to form a ‘combined COR’ for the ADR.

### REFERENCE TO RELATED APPLICATION

This application claims priority from U.S. Provisional Patent Application Serial No. 60/379,462, filed May 10, 2002, the entire content of which is incorporated herein by reference.

### FIELD OF THE INVENTION

This invention relates generally to prosthetic implants and, more particularly, to artificial disc replacement (ADR) devices including springs and other improvements.

### BACKGROUND OF THE INVENTION

Artificial disc replacements (ADRs) are frequently made of hydrogels or metal and rubber. Hydrogel ADRs generally surround the hydrogel core with a flexible constraining jacket, as shown in PCT/USOO/80920, WO 00/59412.

Unfortunately, the flexibility of the hydrogel and the constraining jacket allow hydrogel ADRs to change shape and extrude through defects in the annulus through which the ADR was inserted, for example. Metal and rubber ADRs often fail at the metal-rubber interface. The rubber fails with the high shear stresses or the rubber separates from the metal with shear stress.

There does exist issued patents that relate to enclosing or sealing hydrogel materials. Of interest is U.S. Pat. No. 6,022,376, which teaches a hydrogel enclosed by a fluid permeable bag. However, the fluid bag does little to protect the hydrogel from shear stress, and the rough texture of the bag may cause hydrogel wear from friction.

U.S. Pat. No. 5,002,576 teaches an elastomer enclosed by rigid cover plates and a corrugated tube. The elastomer is sealed from fluids of the body. The corrugated tube allows movement of the cover plates. The corrugated tube may reduce shear forces on the elastomer. U.S. Pat. Nos. 5,865,846; 6,001,130; and 6,156,067 teach a spherical articulation between ADR EPs and an elastomer. The elastomer may be sealed within the ADR EPs. An annular gasket may reduce shear forces on the elastomer. U.S. Pat. No. 5,893,889 teaches an elastomer that is sealed between ADR EPs. The device uses a ball and socket feature to reduce shear on the elastomer. U.S. Pat. No. 6,063,121 incorporates X-shaped wires into the '889 device to reduce rotation.

### SUMMARY OF THE INVENTION

This invention is broadly directed to the use of one or more springs in an artificial disc replacement (ADR) to provide natural movement and extend life of the springs or ADR. In the preferred embodiment, the springs articulate with at least one convex or concave surface on an endplate (EP) of the ADR. More particularly, the spring or springs may articulate with, or connect to, concave or convex articulating EP components. In various alternative embodiments, the ADR EPs may include features that impinge or otherwise limit maximum load on a spring or on multiple springs.

The springs may be disposed in cylinders, over posts, or otherwise constrained. For example, spring posts with convex or concave surfaces may articulate with corresponding concave or convex components, or concave or convex surfaces, on ADR EPs. Also, the center of rotation (COR) of the preferably spherical joint may vary vertically by compression and expansion of a compressible component. Alternatively, the ADR may include multiple, separate CORs that cooperate simultaneously to form a ‘combined COR’ for the ADR.

### BRIEF DESCRIPTION OF THE DRAWINGS

FIG. 1A is a side view of a contained artificial disc replacement (ADR) of the present invention;

FIG. 1B shows the cross-section of the device of FIG. 1A;

FIG. 1C is an exploded view of the device of FIGS. 1A and 1B;

FIG. 1D is a top view of FIGS. 1A-1C in position between a pair of adjacent vertebrae;

FIG. 1E shows the device in a dehydrated state;

FIG. 1F shows the device in a hydrated/expanded state;

FIG. 2A shows an ADR according to the present invention disposed symmetrically between adjacent vertebrae;

FIG. 2B illustrates an asymmetrical configuration;

FIG. 3A illustrates a device dehydrated for insertion between the vertebrae;

FIG. 3B illustrates the device expanded after insertion and hydration;

FIG. 4A shows the device of the present invention with endplates in position;

FIG. 4B is a cross-section of FIG. 4A;

FIG. 5A is a simplified side view of an alternative embodiment of an ADR;

FIG. 5B shows a cross-section of the more encapsulated device showing channels for facilitate fluid transfer;

FIG. 5C is a cross-section showing the hydrogel in a desiccated state;

FIG. 5D is a cross-section showing the hydrogel in a hydrated, expanded form;

FIG. 5E shows the side view of the device in place between upper and lower vertebrae;

FIG. 5F is an anterior-posterior view of the device in place;

FIG. 6A is a side-view of the device of FIG. 5A with inferior and superior end plates attached to the respective vertebrae;

FIG. 6B is an anterior-posterior view of the device of FIG. 6A in position;

FIG. 7A is an anterior-posterior view of in partial cross-section of an ADR incorporating multiple cylinders;

FIG. 7B is a side-view, also in partial cross-section;

FIG. 7C is an axial cross-section of a device containing a central guide cylinder surrounding six pistons;

FIG. 7D shows two embodiments with multiple cylinders;

FIG. 8A is a coronal/sagittal cross-section of the cylinders according to the present invention;

FIG. 8B is an illustration of two vertebrae in extension;

FIG. 9 shows an embodiment with the peg projecting from the posterior aspect of the inferior surface of the upper plate;

FIG. 10A shows a further alternative embodiment of the present invention;

FIG. 10B is a frontal view in cross-section showing partial cushioning;

FIG. 10C is a frontal cross-sectional view illustrating full cushioning;

FIG. 11A is a top-down view of an embodiment showing opposing retaining cylinders on either side of a central resilient member;

FIG. 11B is a side-view drawing in cross-section showing partial cushioning of the device of FIG. 11A;

FIG. 11C is a side-view drawing in partial cross-section illustrating the embodiment of FIGS. 11A and 11B;

FIG. 12A shows an anterior-posterior view of the embodiment of the invention wherein the end plates of ADR may contain hollow keels on the vertebral side;

FIG. 12B is a lateral view of FIG. 12A;

FIG. 12C is a top-down view illustrating the bone ingrowth area of FIG. 12A;

FIG. 13 is a cross-section of an embodiment with multiple pistons connected to the top plate via a rod;

FIG. 14A is a cross-section illustrating an anterior-posterior view of two pedicle screws;

FIG. 14B is a cross-sectional lateral view of the embodiment of FIG. 14A;

FIG. 15A is a side-view of a pedicle screw having an axle to receive a shock absorber according to the present invention;

FIG. 15B is a close-up of the shock absorber mechanism associated with a pedicle screw embodiment of FIG. 15A;

FIG. 16 is a cross-sectional view of a tibial component according to the present invention;

FIG. 17 is a drawing which shows how a locking component may be incorporated in the design;

FIG. 18 is a side-view cross-section of a tibial component for a knee replacement;

FIG. 19 is a side-view drawing of an embodiment illustrating the way in which the invention may be applied to the hip;

FIG. 20A is a lateral view of a variation including a superior ADR EP;

FIG. 20B is a view of the top of the convex caps and the bottom ADR EP of the embodiment of the ADR shown in FIG. 20A;

FIG. 20C is a sagittal cross section through an embodiment of the ADR similar to that drawn in FIG. 20A;

FIG. 20D is a sagittal cross section through the ADR drawn in FIG. 20C;

FIG. 21A is a lateral view of an alternative embodiment of an ADR according to the present invention;

FIG. 21B is a sagittal cross section of an embodiment of the ADR similar to that drawn in FIG. 21A;

FIG. 22 is a lateral view of an alternative embodiment of an ADR;

FIG. 23 is a sagittal cross section through an alternative embodiment wherein the caps and springs are contained in cylinders;

FIG. 24 is a lateral view of an alternative embodiment wherein the top of the spring caps are concave rather than convex as drawn in FIG. 20A;

FIG. 25A is a lateral view of an alternative embodiment illustrating the use of a C-shaped spring that cooperates between convex projections from the ADR EPs;

FIG. 25B is a sagittal cross section through the embodiment of the ADR shown in FIG. 25A;

FIG. 25C is a view of the top of the springs and inferior ADR EP shown in FIG. 25A;

FIG. 25D is a sagittal cross section through an alternative embodiment of the ADR drawn in FIG. 25A;

FIG. 25E is a sagittal cross section through the embodiment of the ADR drawn in FIG. 25D; and

FIG. 26 is a lateral view of the spine and an alternative embodiment including spring caps that articulate with a vertebral endplate.

### DETAILED DESCRIPTION OF THE INVENTION

This invention addresses and solves problems associated with artificial disc replacement (ADR) devices and joint-related components, including those associated with total-knee and hip arthroplasty, by effectively combining the advantages of hydrogels and other compressible/resilient materials while minimizing shear stresses. When applied to an ADR, the invention also minimizes the risk of extrusion.

Hydrogels are used in the preferred embodiments. U.S. Pat. Nos. 5,047,055 and 5,192,326, both incorporated by reference, list some of the applicable hydrogels. The small size of the desiccated hydrogel facilitates insertion, after which the hydrogel imbibes fluids and expands. Other non-hydrogel compressible and/or resilient materials may alternatively be used, including elastomers, shape-memory polymers, which would increase in height after they are inserted. As another example of many, non-hydrogel polymers such as acrylics may be used which change shape with a change in temperature. Thus, as used herein, the term “hydrogel” should be taken to include other resilient/compressible materials.

According to the invention, the hydrogels are protected from shear stress, thereby extending longevity. In particular, the hydrogel is contained, constrained or enclosed within a cavity or cylinder which may include one or more pistons. The hydrogel provides cushioning, while the metal pistons facilitate articulate either directly or indirectly with bone surfaces. Thus, the invention offers the advantages of metal-on-metal while providing for cushioning. The hydrogels allow for physiologic tension adjustment since they can change size based upon imbibing fluid and the pressure on the hydrogel. Thus, the hydrogel component of the device can change height to balance the forces against the surrounding tissues.

The cylinder and piston would likely be made of metal such as stainless steel, titanium, chrome cobalt, or other biocompatible metal or ceramic alloy. Surfaces to promote bone ingrowth could be used on the covers. The hydrogel embodiments may incorporate channels for the diffusion of fluids in and out of the cylinder. Optional permeable membranes can also be used to prevent extrusion of the hydrogel through the channels. The permeable membrane traps the hydrogel but allows fluids to move freely across the membrane.

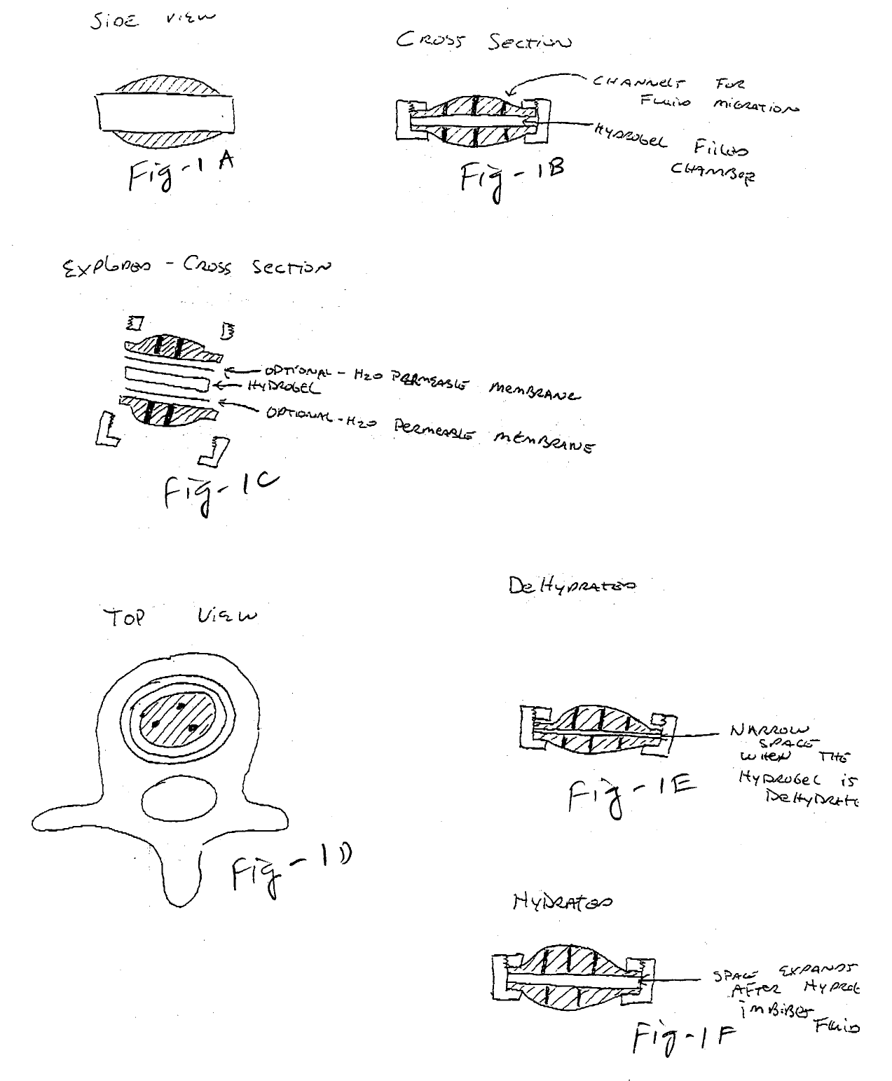

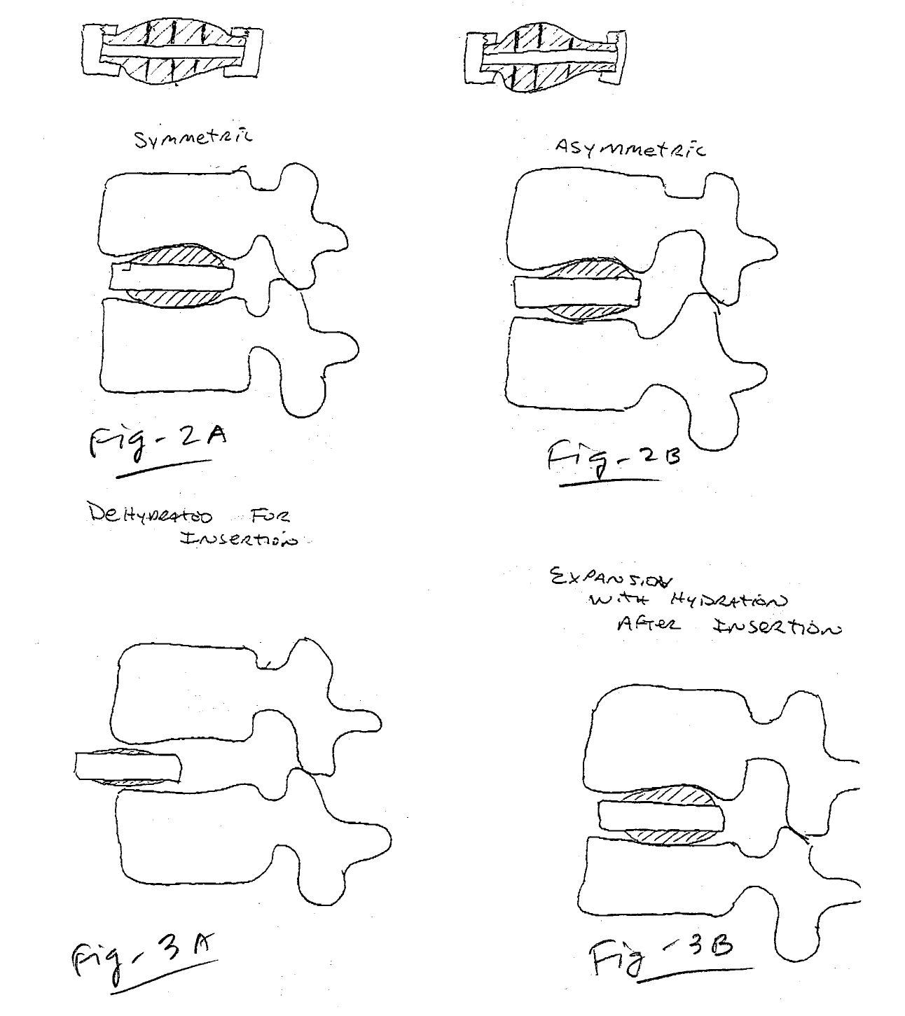

FIG. 1A is a side view of a contained artificial disc replacement (ADR) according to the invention. FIG. 1B is a drawing that shows cross-section of the device of FIG. 1A. FIG. 1C is an exploded view of the device of FIGS. 1A and 1B. FIG. 1D is a top view of FIGS. 1A-1C in position between a pair of adjacent vertebrae. FIG. 1E shows the device in a dehydrated state; FIG. 1F shows the device in a hydrated/expanded state.

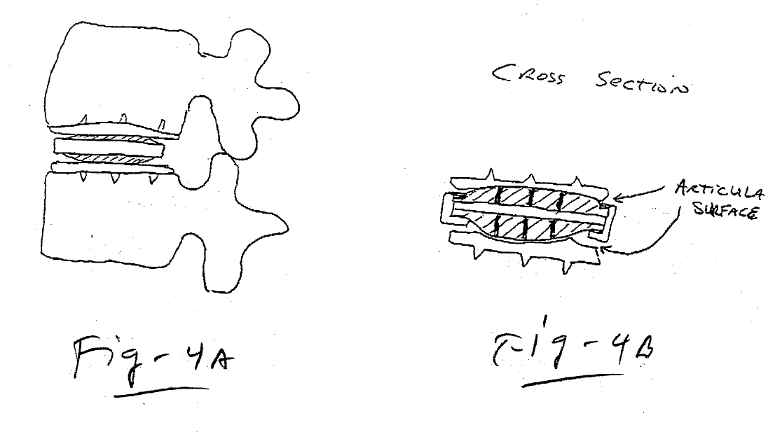

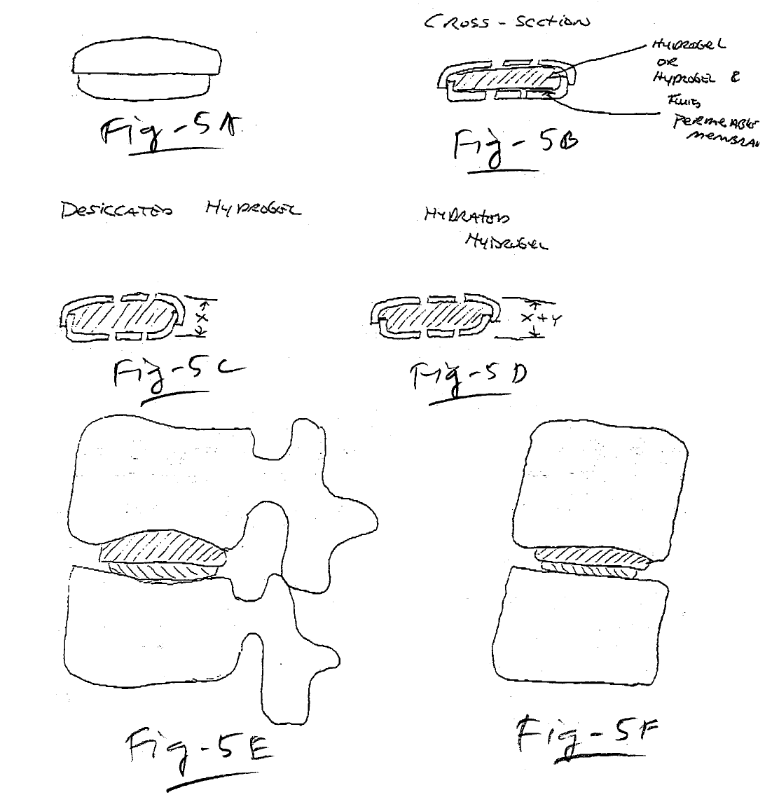

Devices according to the invention, regardless of disposition in the body, may be placed symmetrically or asymmetrically. FIG. 2A shows an ADR according to the invention disposed symmetrically between adjacent vertebrae. FIG. 2B illustrates an asymmetrical configuration. FIG. 3A illustrates a device dehydrated for insertion between the vertebrae and FIG. 3B illustrates the device expanded after insertion and hydration. As shown in FIG. 4, endplate covers may be provided in conjunction with the contained hydrogel ADR according to the invention. FIG. 4A shows the device and endplates in position. FIG. 4B is a cross-section.

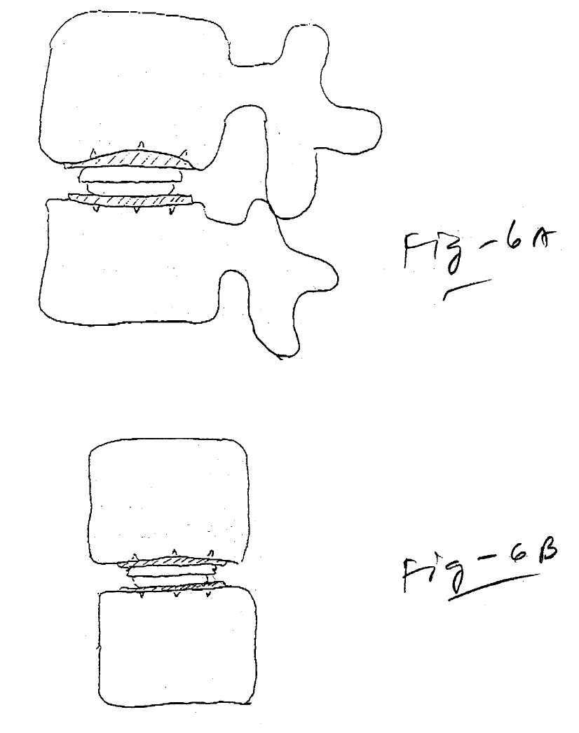

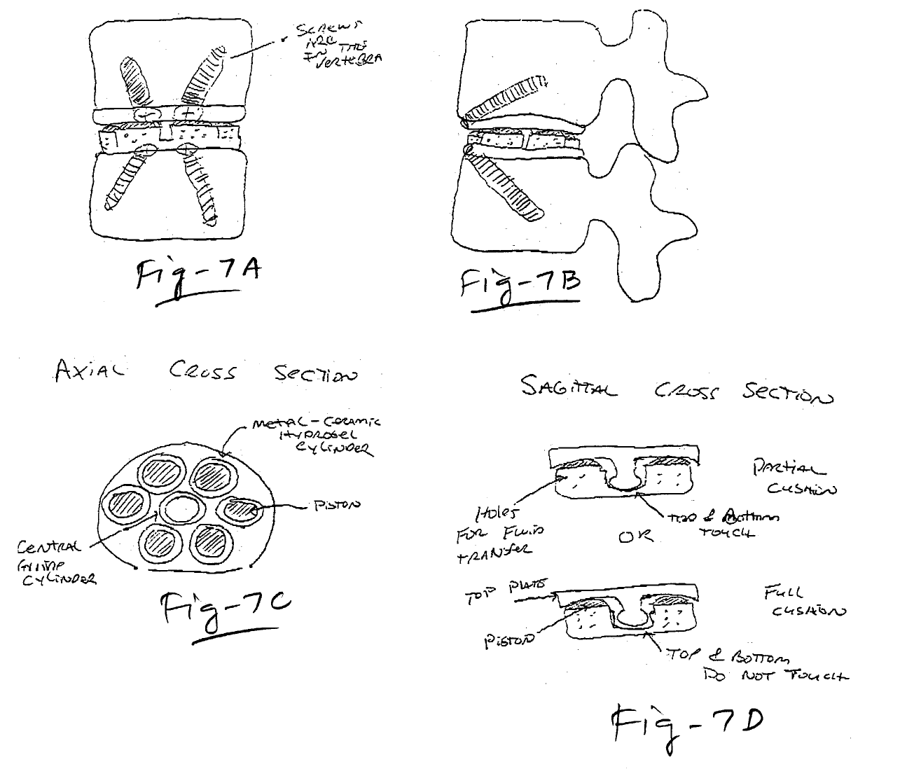

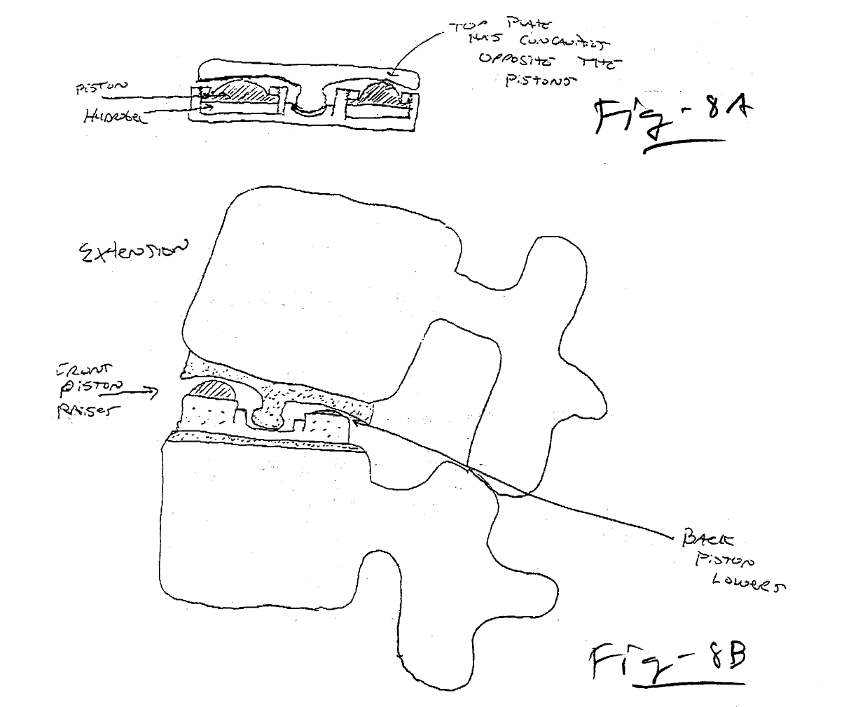

FIG. 5A is a simplified side view of an alternative ADR according to the invention, wherein the hydrogel is further encapsulated. FIG. 5B is a cross-section of the more encapsulated device showing channels for facilitate fluid transfer. FIG. 5C is a cross-section showing the hydrogel in a desiccated state. FIG. 5D is a cross-section showing the hydrogel in a hydrated, expanded form. FIG. 5E shows the device in place between upper and lower vertebrae from a side view. FIG. 5F is an A-P of the device in place. FIG. 6A is a side-view of the device of FIG. 5, with inferior and superior end plates attached to the respective vertebrae. FIG. 6B is an A-P view of the device of FIG. 6A in position.

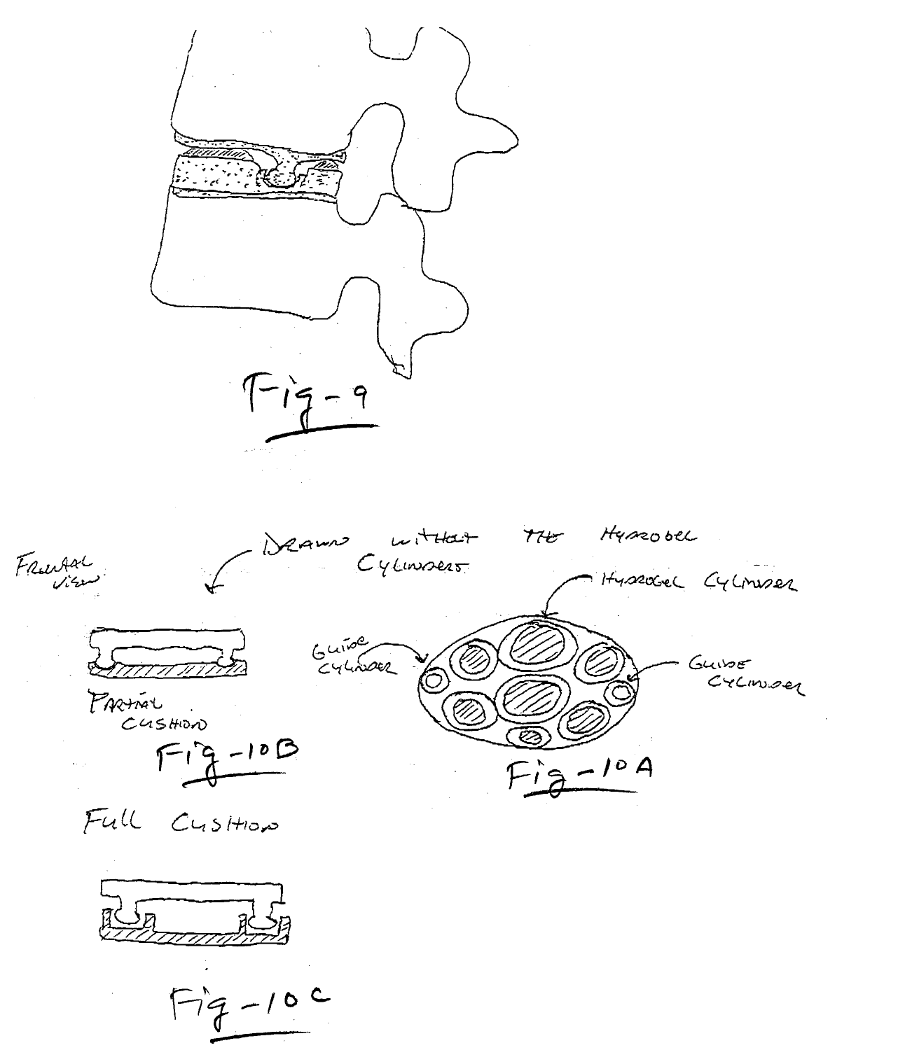

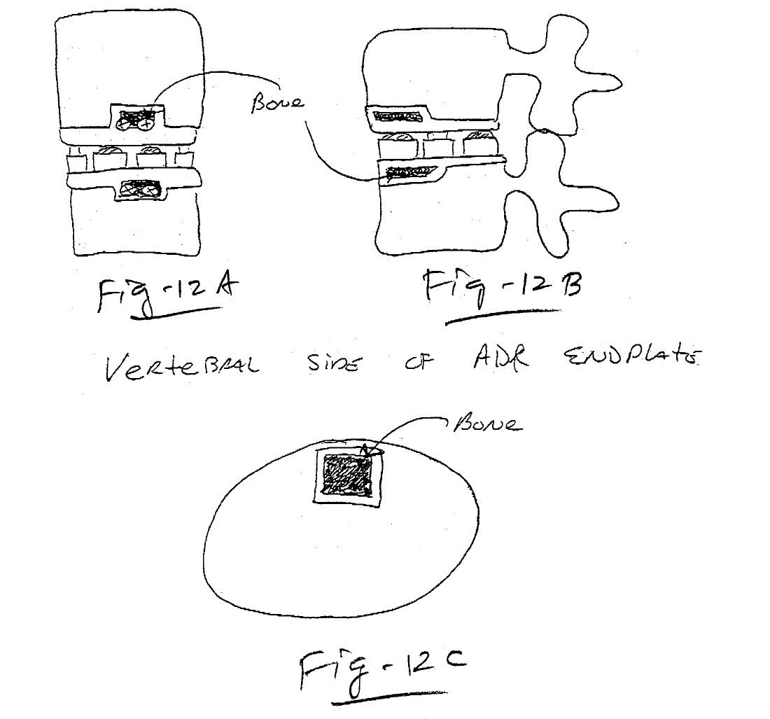

The invention may also include two or more cylinders. Adding cylinders reduces the tendency of a single assembly to tilt when pressure is applied in an eccentric fashion. FIG. 7A is an A-P view of in partial cross-section of an ADR incorporating multiple cylinders. FIG. 7B is a side-view, also in partial cross-section. FIG. 7C is an axial cross-section of a device containing a central guide cylinder surrounding six pistons. It will be appreciated that more or fewer guide cylinders and/or pistons may be used as shown, for example, in FIG. 10.

FIG. 7D shows two embodiments with multiple cylinders. In the partial cushion embodiment (upper drawing), the spherical end of the peg projecting from the top plate rests against and is partially supported by a concavity in the lower plate. In the full cushion embodiment (lower drawing), the peg projecting from the top plate fits into a restraining cylinder. The peg form the top plate does not rest against the bottom plate in this embodiment. In either case, the end of the peg is preferably spherical to allow angular motion between the two plates.

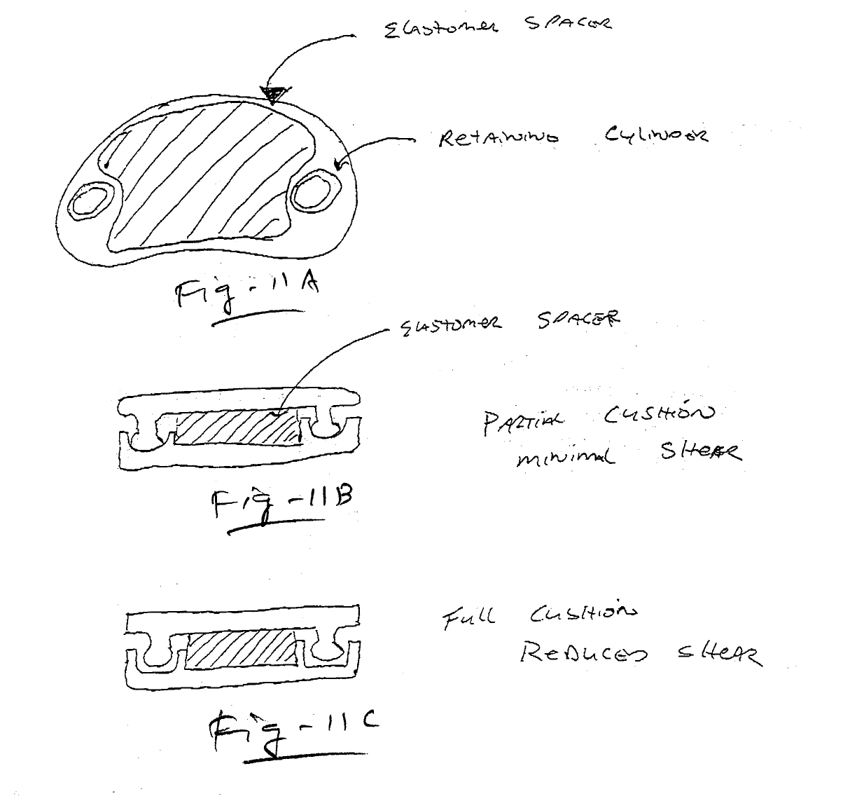

FIG. 8A is a coronal/sagittal cross-section of the cylinders according to this embodiment of the invention. FIG. 8B is an illustration of two vertebrae in extension, showing the way in which the front piston is raised and the back piston is lowered. Note that the peg that projects from the lower portion of the upper plate need not be central in location. FIG. 9 shows an embodiment with the peg projecting from the posterior aspect of the inferior surface of the upper plate. Posterior peg placement allows a larger anterior cylinder. The larger anterior cylinder may be better at handling the larger forces placed on the anterior portion of the disc replacement during spinal flexion.

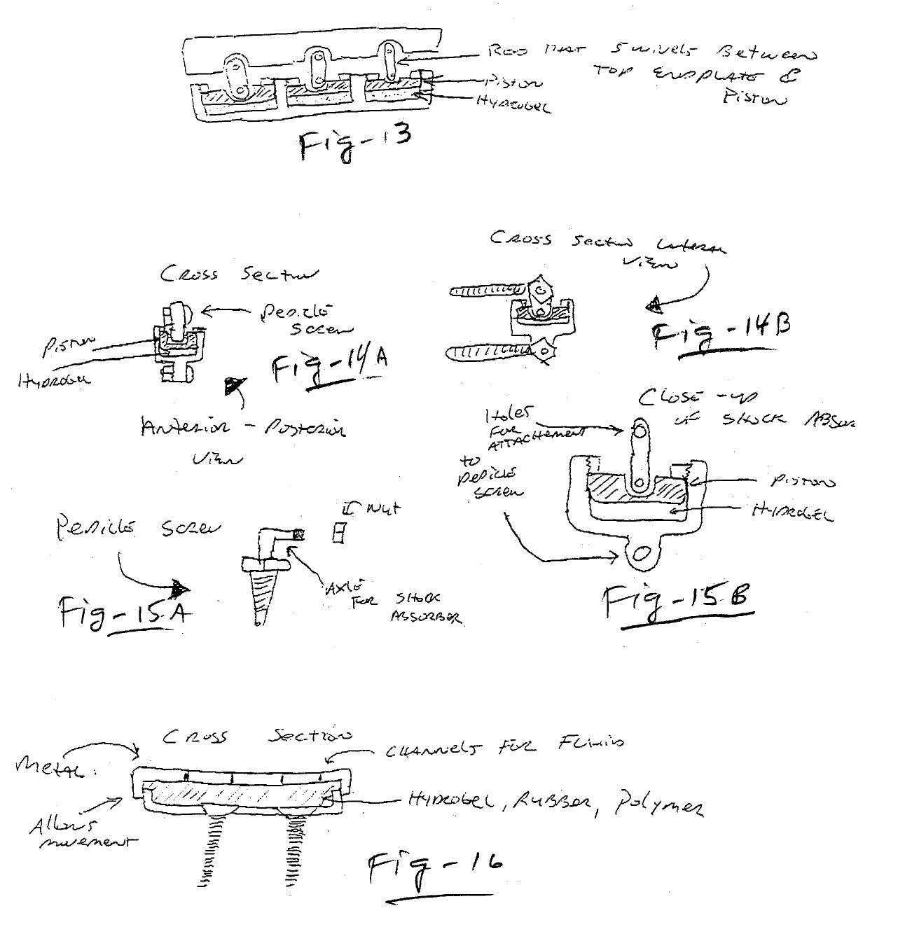

FIG. 10 is a drawing which shows an alternative arrangement wherein multiple guide cylinders are used at the periphery as opposed to a central location. Among other advantages, this may help to prevent rotatory subluxation of the top component relative to the bottom component while allowing more area centrally for the hydrogels/polymer cylinders. FIG. 10A is a top cross-section view of an embodiment showing multiple peripheral cylinders and additional internal hydrogel chambers. FIG. 10B is a frontal view in cross-section showing partial cushioning. FIG. 10C is a frontal cross-sectional view illustrating full cushioning. Two or more retaining cylinders may also be used to reduce the shear on the solid piece of silicone rubber, elastomer or hydrogel-type material. FIG. 11A is a top-down view of an embodiment showing opposing retaining cylinders on either side of a central resilient member. FIG. 11B is a side-view drawing in cross-section showing partial cushioning of the device of FIG. 11A. FIG. 11C is a side-view drawing in partial cross-section illustrating the embodiment of FIGS. 11A and 11B providing a full cushioning and reduced shear capability.

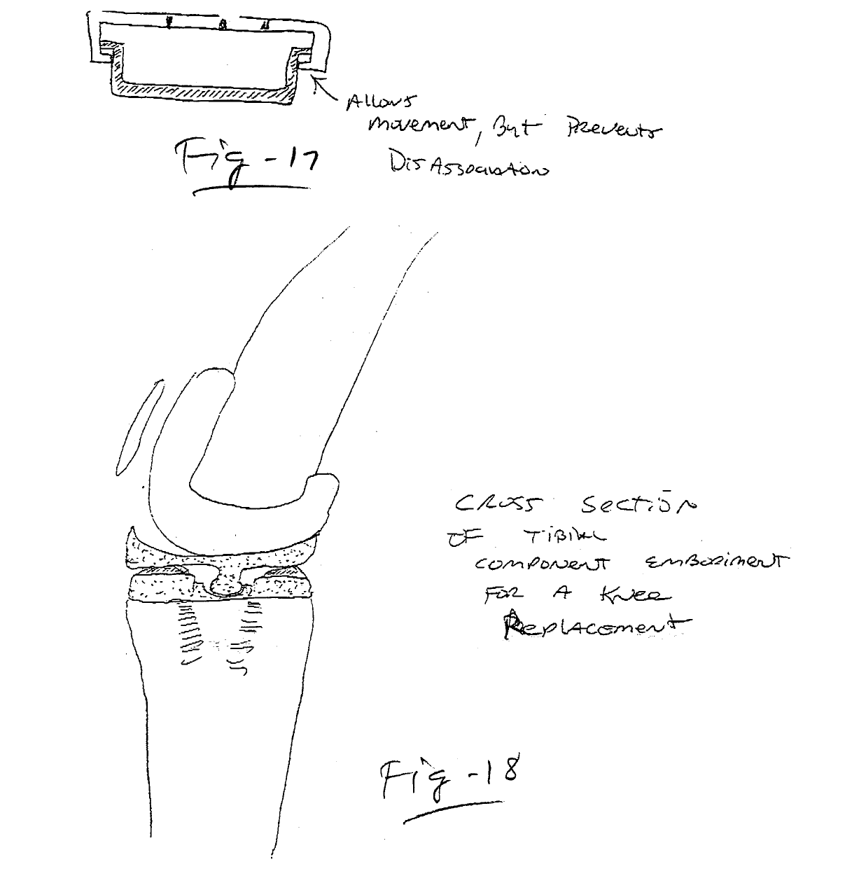

Reference is now made to FIG. 12A, which is an A-P view of the embodiment of the invention wherein the end plates of ADR may contain hollow keels on the vertebral side. FIG. 12B is a lateral view and, FIG. 12C is a top-down view illustrating the bone ingrowth area. The vertebrae would be osteotomized to make room for the keels. The bone from the osteomity sites would be morselized and placed inside the hollow keels. The morselized bone would promote ingrowth into the end plates of the ADR, much like hollow cages promote bone ingrowth.

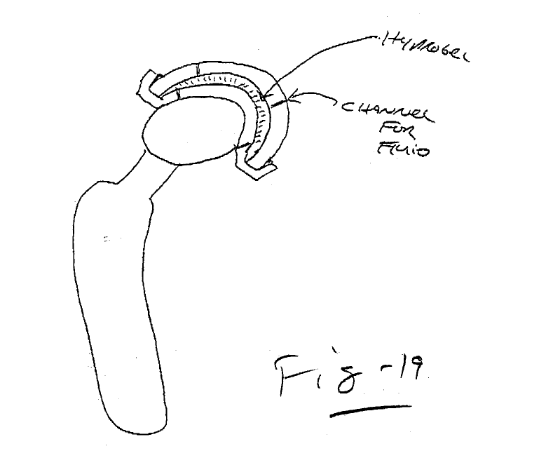

FIG. 13 is a cross-section of an embodiment with multiple pistons connected to the top plate via rod, much like the design of rods that connect pistons to a crankshaft in an engine. The shock absorber concept according to this invention may also be used with respect to vertebral shock absorbers. FIG. 14A is a cross-section illustrating an A-P view of two pedicle screws coupled in this way. FIG. 14B is a cross-sectional lateral view of the embodiment of FIG. 14A. FIG. 15A is a side-view of a pedicle screw having an axle to receive a shock absorber according to the invention. FIG. 15B is a close-up of the shock absorber mechanism associated with a pedicle screw embodiment.

The cylinders could be made of ceramic, metal, or metal lined with ceramic. The pistons could also be made of metal, ceramic, alloys and so forth. In any case, the articulation of the top and bottom plates is preferably metal-to-metal or ceramic-to-metal, both of which are presumably superior to metal-to-polyethylene articulations. Furthermore, hydrogels, shape memory polymers, or other polymers within the cylinder provide a cushion, or dampen the forces across the plates.

Polymers of different durometers could be used in cylinders in different locations. For example, the polymers in the posterior cylinders could be less compressible and therefore help resist extension of the spine. The cylinders could also use liquids with baffles to dampen motion. That said, hydrogels or polymers have the benefit of functioning without a water-tight cylinder piston unit. Indeed, as mentioned previously, the cylinders or the pistons may contain holes to allow fluid movement in the hydrogel configurations.

As discussed above, this invention is not limited to the spine, but may be used in other joint situations such as the knee and hip, which typically use polyethylene bearing surfaces on the acetabulum or proximal tibia. Problems related to polyethylene wear are well known to orthopedic surgeons. Although metal-on-metal and ceramic-on-ceramic total hips have been developed to reduce the problems associated with poly wear, such designs do not provide shock-absorbing capacity. For example, excessive force form tight ligaments about the knee or hip may reduce the size of the hydrogel, thus reducing the tension on the ligaments. Conversely, loose ligaments will cause the hydrogel to swell, thus increasing the tension on the loose ligaments. Although hydrogels are used in this preferred embodiment as well, other elastomers and polymers including shape memory polymers may alternatively be used.

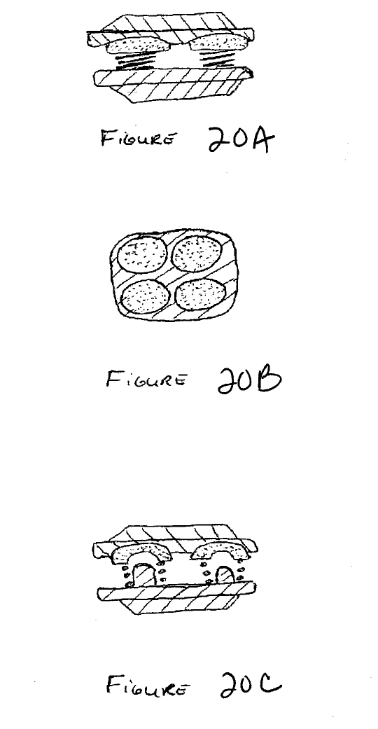

FIG. 16 is a cross-sectional view of a tibial component according to the invention. As discussed above, channels are used for fluid transfer, and these may be located around the periphery, or near the center, rather than in the weight-bearing area. FIG. 17 is a drawing which shows how a locking component may be incorporated in the design which allows movement while, at the same time, prevent disassociation. A similar design may be used for other prosthetic components, including a patella button. FIG. 18 is a side-view cross-section of a tibial component for a knee replacement utilizing a central guide and peripheral pistons, much like the vertebral embodiments discussed with reference to FIGS. 7-11, in particular.

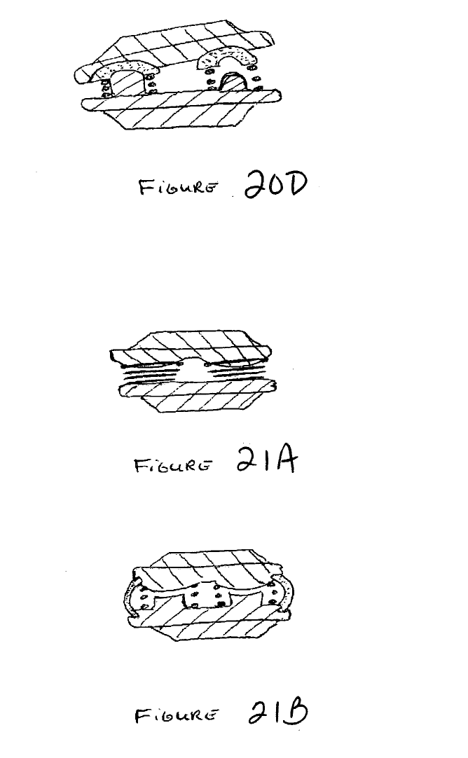

FIG. 19 is a side-view drawing of an embodiment illustrating the way in which the invention may be applied to the hip. As shown in the drawing, an inner cup would be used with respect to the acetabulum, along with an outer bearing surface with a hydrogel/elastomeric or other polymeric material being used therebetween. Particularly with regard to a hydrogel configuration, one or more channels for fluid transfer may be provided.

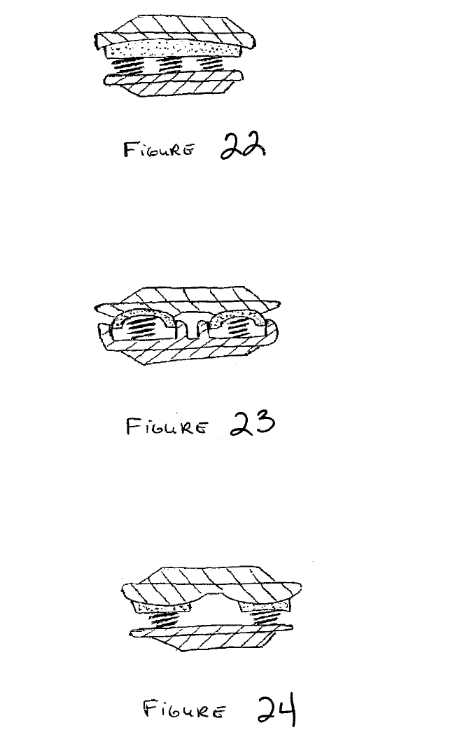

FIG. 20A is a lateral view of a variation including a superior ADR EP that articulates with convex caps which, in turn, articulate with, or are connected to, springs. The articulation between the ADR EP and the caps reduces shear on the springs and on the connection of the springs to the surrounding components. FIG. 20B is a view of the top of the convex caps and the bottom ADR EP of the embodiment of the ADR drawn in FIG. 20A. Any number of springs and caps can be used in the novel ADR. For example, the ADR could use one to twenty springs or more.

FIG. 20C is a sagittal cross section through an embodiment of the ADR similar to that drawn in FIG. 20A. The inferior ADR EP in FIG. 20C has posts that hold the springs in position. FIG. 20D is a sagittal cross section through the ADR drawn in FIG. 20C. The upper ADR EP is tilted with respect to the lower ADR EP as would be seen with spinal movement. The spring on the left is compressed. The post from the inferior ADR EP is articulating with the spring cap. Articulation between the spring cap and the post, limit the amount of compression applied to the spring. Movement occurs through the articulation between the spring cap and the upper ADR EP, and between the spring cap and the post from the lower ADR EP.

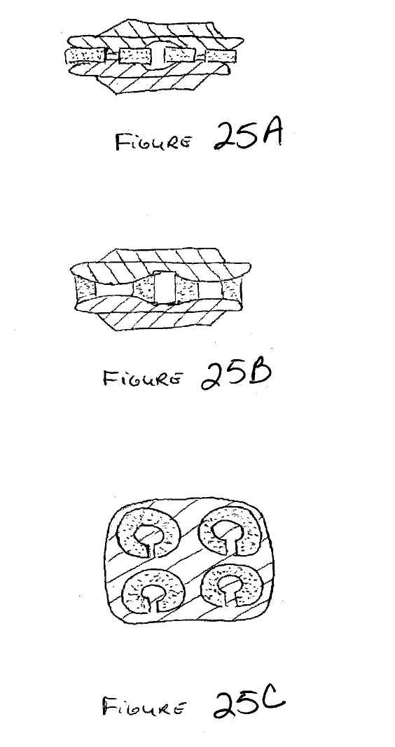

FIG. 21A is a lateral view of an alternative embodiment of an ADR according to the invention, wherein the springs articulate directly with the ADR EPs. The superior ADR EP has convex surfaces that articulate with the springs. The lower ADR EP could have similar convex surfaces. Alternatively, the springs could be connected to or articulate with a flat surface on the lower ADR EP.

FIG. 21B is a sagittal cross section of an embodiment of the ADR similar to that drawn in FIG. 21A. The springs surround posts from the inferior ADR EP. The surface on the top of the post is concave to articulate with the convex projections from the upper ADR EP. The ADR also has an optional component to seal the springs and the articulating surfaces from the body. The seal traps debris from the articulating surfaces. The seal can also be used to contain a lubricating fluid. Various oils or other suitable fluids or gels could be used inside the ADR.

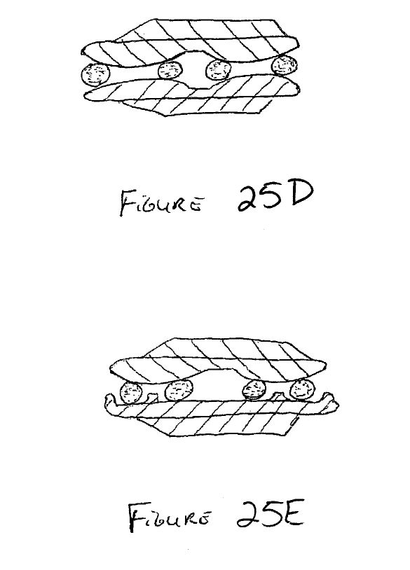

FIG. 22 is a lateral view of an alternative embodiment of an ADR, wherein multiple springs cooperate with a single cap. FIG. 23 is a sagittal cross section through an alternative embodiment wherein the caps and springs are contained in cylinders. FIG. 24 is a lateral view of an alternative embodiment wherein the top of the spring caps are concave rather than convex as drawn in FIG. 20A.

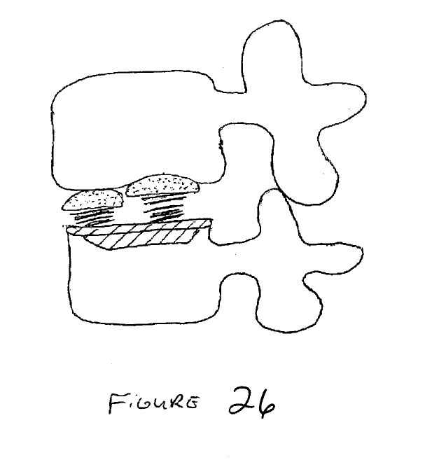

Springs of other types can be used in this and the other embodiments of this invention. For example, FIG. 25A is a lateral view of an alternative embodiment illustrating the use of a C-shaped spring that cooperates between convex projections from the ADR EPs. FIG. 25B is a sagittal cross section through the embodiment of the ADR drawn in FIG. 25A. FIG. 25C is a view of the top of the springs and inferior ADR EP drawn in FIG. 25A. FIG. 25D is a sagittal cross section through an alternative embodiment of the ADR drawn in FIG. 25A. The C-shaped springs are preferably circular in cross section. FIG. 25E is a sagittal cross section through the embodiment of the ADR drawn in FIG. 25D. The inferior ADR EP has posts to hold the springs in position. The springs articulate with the flat surface of the inferior ADR EP.

FIG. 26 is a lateral view of the spine and an alternative embodiment including spring caps that articulate with a vertebral endplate. The use of independent springs allow the ADR to better conform to the vertebral endplate. For example, one or more of the springs can extend more completely to fill concavities within the vertebral endplates.

I claim:

1. An artificial disc replacement (ADR), comprising:
a pair of opposing plates;
at least one spring disposed between plates to urge them apart; and
a concave or convex surface on one of the plates where the spring contacts that plate.

2. The ADR of claim 1, further including one or more features that impinge or otherwise limit the load on the spring or springs.

3. The ADR of claim 2, including a spring disposed in a cylinder.

4. The ADR of claim 2, including a spring disposed over a post.

5. The ADR of claim 1, wherein the point where the spring contacts the concave or convex surface results in a joint having a center of rotation.

6. The ADR of claim 5, wherein the joint is spherical.

7. The ADR of claim 5, including a plurality of springs, each forming a joint having a center of rotation.

8. The ADR of claim 7, wherein the centers of rotation cooperate to form an overall center of rotation for the ADR.
